(19) United States
(12) Patent Application Publication
(10) Pub. No.: US 2001/0031268 A1
(43) Pub. Date: Oct. 18, 2001

(54) Antigen preparations

(76) Inventors: Thomas John Baldwin, Nottingham (GB); Paul Williams, Nottingham (GB); Simon Swift, Leicestershire (GB); Cornelia Suzanna McLean, Cambridge (GB); Christopher Lee Holmes, Herfordshire (GB)

Correspondence Address: KLARQUIST SPARKMAN CAMPBELL LEIGH & WHINSTON, LLP, Suite 1600, One World Trade Center, 121 S.W. Salmon Street, Portland, OR, 97204-2988, US

(21) Appl. No.: 09/740,048

(22) Filed: Dec. 18, 2000

Related U.S. Application Data
(60) Provisional application No. 60/172,485, filed on Dec. 17, 1999.

(51) Int. Cl. A61K 39/095; A61K 39/108

(52) U.S. Cl. 424/249.1; 424/257.1

(57) ABSTRACT

Bacterial antigen preparations for use as live or killed immunogens and vaccines can be produced by culture of bacteria such as Neisseria in medium comprising norepinephrine or other catechol-group-containing growth inducer of bacterial growth, harvesting and pharmaceutical formulation. The antigen preparations can comprise bacterial protein(s) inducible by norepinephrine.

### FIELD OF THE INVENTION

This invention relates to the preparation and use of antigens, e.g. for pharmaceutical use in connection with bacterial vaccines, for example vaccines against Neisseria, e.g. Neisseria meningitidis, e.g. of Group B or C, and vaccines against other pathogens, such as E. coli, e.g. E. coli of type 0157 or of a type immunologically crossreactive therewith. In further aspects the invention also relates to antigenic preparations that can be so produced, and to pharmaceutical preparations, such as live attenuated or killed vaccines, comprising them.

### BACKGROUND

Many immunogens and vaccines related to bacterial diseases are known or have been proposed, including immunogens and vaccines that can be made from Neisseria and E. coli among other bacterial types.

For example, specification WO 98/56901 (Medical Research Council: Baldwin et al) describes attenuated bacteria, e.g. Neisseria meningitidis, for use inter alia as live vaccine material, in which besides an attenuating mutation the native fur (ferric uptake regulation) gene or homologue thereof is modified such that the expression of the fur gene product (or its homologue) is regulated independently of the iron concentration in the environment of the bacterium.

A live attenuated Neisseria vaccine has also been proposed by C Tang et al. in Vaccine 17 (1999) pp 114-117.

Another technique for producing vaccine against Neisseria meningitidis is described in U.S. Pat. No. 5,597,572 (Centro Nacional De Biopreparados: C C Huergo et al.), disclosing a vaccine against infection caused by Group B Neisseria meningitidis microorganism, which comprises a purified protein antigenic complex weighing from 65 to 95 kDa, vesicles, and capsular polysaccharide; the vaccine is extracted from the cell membranes of the live microorganisms using detergent and enzyme.

Induction of iron-regulated proteins during normal growth of Neisseria meningitidis in a chemically defined medium has been reported by MC Brandileone et al, Rev Inst Med Trop Sao Paolo (1994) (Jul-Aug) 36(4): pp 301-310, which reported culture in the presence of EDDA (ethylene diamine di-ortho-hydroxyphenyl-acetic acid) in iron-deficient Catlin medium containing EDDA and hemin, to yield outer membrane vesicles expressing iron regulated proteins.

In A Pettersson et al. (in Antonie van Leeuwenhoek (1997) 71: pp 129-136) a protein designated lactoferrin-binding protein A (LbpA) is discussed as an example of a receptor produced by Neisseria meningitidis when grown under iron starvation, which has affinity for an iron-storage protein: the paper discusses the vaccine potential of LbpA and FrpB proteins encoded by Neisseria meningitidis.

It is also known that many bacteria secrete small-molecule organic iron-chelators called siderophores. These bind ferric iron with high affinity and are able to compete with other iron-binding proteins for iron. Iron-siderophore complexes can be bound by specific receptors on the cell surface of bacteria, including some which do not synthesise siderophores of their own, and can be intemalised to provide iron for growth of the bacterial cell.

Bacteria attenuated by auxotrophic mutations are also known, as is the application of certain of them as live attenuated vaccines. Examples of auxotrophic mutations are described in certain of the following publications:

EP 0 322 237 (Wellcome: G Dougan & S N Chatfield) describes bacteria attenuated by a non-reverting mutation in at least two aro genes, and use of the attenuated bacterium as a vaccine.

U.S. Pat. No. 5,210,035 (Leland Stanford University: BAD Stocker) discloses a method of preparing a live attenuated bacterial vaccine, for example a Neisseria vaccine, by blocking at least one biosynthetic pathway, such as the aro pathway, by at least two non-reverting mutations which involve at least 5 nucleotides each.

WO 94/05326 (University of Saskatchewan: B J Allen & A A Potter) describes bacterial vaccines attenuated by, for example, mutations in the pyr pathway or by an iron metabolism mutation.

Bacterial endotoxin mutants, for example of Neisseria, are described in WO 99/10497 (De Staat de Nederlanden: P A Van Der Lay & L J J M Steeghs) which discloses the use as vaccines of live gram-negative mucosal bacteria which retain outer membrane proteins but lack endotoxic LPS, such as Lipid A mutants.

EP 0 624 376 (American Cyanamid: G W Zlotnik) describes a method for removing bacterial endotoxin from gram-negative cocci, e.g. Neisseria, and the use of such endotoxin-depleted outer membranes and soluble outer membrane proteins as vaccines.

Use of non-encapsulated bacterial mutants as vaccines has been described in WO 93/10815 (Centre for Innovated Technology: T J Inzana). WO 97/49416 (Virginia Tech: T J Inzana & C Ward) also discloses deletion of capsule-encoding genes in bacteria, and use of such mutants, for vaccine purposes.

Bacterial capsule mutants containing GalE mutations, and use of such mutants as vaccines, are described in EP 0 249 449 (Enterovax Research: DM Hone & J A Hackett).

Bacteria which do not express a functional recombinase as a result of a recA mutation, and use of these mutants as vaccines, is disclosed in WO 95/25738 (Vanderbilt University: S A Thompson & M J Blaser).

Stimulatory effects of catecholamine on bacterial growth have been reported and discussed in relation to in-vivo levels of catecholamines and the severity of sepsis in infected humans and animals (M Lyte and S Ernst in Life Sci. 50: pp 203-212). It remains desirable to provide further methods for producing immunogens suitable for use in vaccines against bacterial pathogens, e.g. Neisseria meningitidis, as well as immunogenic preparations producible by such methods, and this is among the aims of the invention.

### SUMMARY AND DESCRIPTION OF THE DISCLOSURE

The present invention is based in part on our observation that bacteria for example Neisseria meningitidis and E. coli (and others which contain a catechol-uptake pathway, e.g. enterobacteriaceae and pseudomonadaceae, e.g. Ps. aeruginosa) can respond in a way not previously known to norepinephrine and related substances, which induce growth and protein expression. A similar response occurs with other inducer agents of which the molecules contain catechol groups. These include per-se known siderophores, which can interact with the catechol-uptake pathway of such bacteria, as further discussed herein. It is believed that this response occurs when iron is supplied to the bacteria by said inducer, e.g. by iron bound to said inducer. According to the present disclosure this effect can be utilised in the culture of such bacteria for preparing antigens such as vaccines.

Thus in one aspect the disclosure provides a method of producing a bacterial antigen preparation. In one embodiment, a method comprises the steps of (a) culturing bacteria selected from the group consisting of Neisseria, E. coli, and other bacteria that possess a catechol-uptake pathway, in a medium which comprises a catechol-group-containing inducer of bacterial growth, whereby said inducer stimulates growth of said bacteria, (b) harvesting from said culture a bacterial antigen, and (c) formulating said antigen with a pharmaceutically acceptable carrier for use as an immunogen. In another aspect, the invention provides an antigen preparation comprising bacterial protein from Neisseria or E. coli or said related bacteria, obtainable by culture of said bacteria according to a process as described herein, so that said preparation contains at least one bacterial protein which can be induced by norepinephrine, e.g. a Neisserial antigen preparation containing a Neisserial protein induced by norepinephrine and having a molecular weight of about 50 kDa, or about 55 kDa, or about 20 kDa.

In this specification, unless the context requires otherwise, “inducer” means a growth-inducing and iron-binding compound that contains a catechol group. Examples include those catechol compounds mentioned herein.

Supply of iron to the bacteria, e.g. by iron bound to said inducer, can be achieved for example in a number of ways:

Preferably, for example, bacteria can be grown in a medium containing (a) an iron source and a further iron chelator, e.g. in the form of an iron chelator bound to iron, and also (b) at least one catechol group-containing inducer, e.g. norepinephrine. It is believed that the inducer agent, e.g. norepinephrine, can compete with the further chelator for the iron and make the iron available to the bacteria.

By “further iron chelator” or “further chelator” in this context is understood a chelator of iron which in itself is not a catechol and hence not one of the catechol group-containing inducers referred to above.

In other embodiments such a further chelator need not be present. All or substantially all of the iron present can be bound to the inducer as mentioned above. The inducer can be added in the form of its iron complex, and the medium can otherwise be iron free.

In certain embodiments, culture of the bacteria can be carried out for example in either of two ways, (a) with an inducer agent in the presence of a low, e.g. limiting, iron concentration from the start of growth, or (b) initially under iron-sufficient conditions, followed by addition of inducer agent and decrease of the iron concentration at a later stage of growth. The inducer can be added at a stage of bacterial growth when the iron concentration of the medium has decreased, e.g. when the medium contains an iron limiting concentration of iron.

In general the additives described herein can be used in desired combinations or individually in many embodiments of the invention.

The response of the micro-organism under culture includes accelerated growth accompanied by the expression of bacterial antigens which either are not expressed, or are expressed to a substantially lesser extent, when such bacteria are cultured under otherwise comparable conditions in the absence of such catechol-group-containing inducer agents, whether under low-free-iron conditions or under iron-replete conditions. Such effects have not been seen in the absence of inducer and when iron in the culture is chelated to restrict free iron concentrations by adding chelators such as EDDA, known for use in carrying out iron-restricted culture of such bacteria, or by the addition of serum iron-binding proteins.

In particular, according to an aspect of the present disclosure, catechol-group-containing compounds such as norepinephrine and other inducers of growth able to interact with the bacterial catechol-uptake pathway, are useful as agents for inducing or promoting the expression of desirable antigens in the culture of bacteria, for example Neisseria or E. coli, for use in the production of live attenuated vaccines, and/or of vaccines based on isolated bacterial vesicles or on preparations of isolated bacterial outer-membrane proteins.

In a further aspect, there is provided a method of preparing an immunogenic preparation of bacterial antigens including an immunogenic amount of at least one inducible bacterial antigen, (e.g. a protein of the bacterial iron-uptake system, or a protein which does not belong to the bacterial iron-uptake system), which method comprises (a) culturing a bacterial strain in the presence of a medium containing an inducer agent as described herein, for example norepinephrine, effective to promote expression or enhanced expression of said bacterial antigen, (b) obtaining from said culture an immunogenic preparation containing said bacterial antigen; and (c) formulating said preparation with a pharmaceutically acceptable carrier for immunogenic use, e.g. for pharmaceutical use as a vaccine.

Also provided are culture media and methods that comprise such inducer additives and also optionally comprise other additives as described herein. The disclosure in further aspects provides antigenic bacterial preparations containing amounts, or elevated amounts, of corresponding induced antigen; and preparations of vesicles and outer membrane proteins which can be derived from cultured bacteria, e.g. in per-se known manner; as well as vaccines prepared therewith.

In certain embodiments, inducer agents as described herein, optionally in combination with other additives described herein, can be used as additives in culture media for bacteria, for example Neisseria and E. coli, for the production of immunogens such as vaccines, to induce or promote the expression of bacterial antigen(s), which can be or include protein(s) other than proteins regulated by the bacterial ferric uptake regulation (fur) gene product.

Suitable inducer agents for this purpose can be selected from catechol-group-containing derivatives, e.g. noradrenalin (norepinephrine), and analogues of norepinephrine; 2, 3-dihydroxybenzoylserine; or enterochelin, which is a trimer thereof, and which has a particularly high affinity for iron, with about 10⁻¹⁶ M dissociation constant; and other siderophores or non-toxic iron-chelators, which can be taken up by the catechol uptake pathway and are otherwise compatible with culture of bacteria such as Neisseria or E. coli. Examples of such other chelators include dopamine; the compound catechol itself; isoprenaline; and DOMA (an oxidation product of norepinephrine). A presently-preferred inducer agent is norepinephrine.

If bacteria are grown in the presence of both a further iron chelator, as mentioned above, and an inducer containing a catechol group as mentioned above, it is believed preferable that the chosen inducer has a higher affinity for iron than the further iron chelator. Suitable further chelators that can be used when norepinephrine is the chosen catechol-group-containing inducer include serum preparations, such as adult bovine serum, or transferrin, e.g. purified human or animal transferrin, e.g. recombinant purified human or animal transferrin, e.g. in commercially available forms.

If necessary and if desired in order to assist the choice of medium constituents, the iron affinity of both the inducer, and a substance proposed to be used as a further chelator as mentioned above, can be measured by determining their respective binding constant (Kd) for iron, using standard methods known in the art, e.g. by equilibrium dialysis with radiolabelled iron.

It has been found for example that conventional nutrient media for the growth of bacteria such as Neisseria or E. coli can be usefully modified for the present invention by including therein an amount of inducer, e.g. norepinephrine, effective to stimulate the expression of inducible components as described herein. Suitable amounts can range from about 10⁻¹⁰ to 10⁻³ M norepinephrine, preferably from about 10⁻⁵ to 10⁻³ M, e.g. up to about 2×10⁻⁴ M, e.g. about 10⁻⁵ M, and in one preferred example about 5×10⁻⁵ M of norepinephrine.

Similar amounts can be used of analogues of norepinephrine, of enterochelin, or of 2,3-dihydroxybenzoylserine, and/or of other non-toxic iron-chelators compatible with culture of bacteria such as Neisseria or E. coli.

The choice of culture media for use in connection with this invention can range from defined growth media containing a source of carbon, nitrogen and amino acids to rich complex growth media. Useful commercially available examples of media are Dulbecco's modified Eagle's medium (DMEM; Sigma) and brain heart infusion (BHI: Oxoid).

In certain embodiments involving Neisseria meningitidis, examples of defined media which can usefully be modified for the purposes of the present invention by adding inducer as mentioned above, include the modified Catlin medium described by J. Fu et al. in Bio/Technology 13, pp 170-174 (1995), and also the Frantz medium described in D. Ivan and J. R. Frantz, J. Bacteriol. 43, pp 757-761 (1942).

When bacteria are grown in medium which contains further iron chelator and also a source of iron, the iron source can be a soluble iron salt. Suitable examples include ferrous or ferric salts, e.g., ferric nitrate, or the chlorides, citrates or sulphates. Under these culture conditions, it can be desirable for the bacterial growth medium to contain an amount of iron in the range about 1 to 10 microM (μM), e.g. about 1 microM. An example of such a medium is the modified Catlin medium of J. Fu et al. (Bio/Technology 13, pp 170-174, 1995), which contains ferric citrate.

It is presently preferred to use in the growth medium sufficient of the further chelator to chelate substantially all of the iron present in the growth medium, e.g. the further chelator can be present at about twice to about ten times the molar concentration of iron, e.g. at about five times the iron concentration. For example, the growth medium can contain about 1 microM Fe, and about five microM of the further chelator.

When bacteria are grown in medium containing: (a) inducer and (b) a source of iron and an further iron chelator (or further iron chelator bound to iron), these components (a) and (b) can be added to the growth medium at various stages, e.g. at the same time as the bacterial inoculum. Alternatively and more preferably, the bacterial inoculum and the iron and further chelator can be added to the growth medium at about the same time, and the inducer can be added at a later stage of bacterial growth, e.g. after about 2-6 divisions of the bacteria.

When the growth medium is a complex rich nutrient medium, e.g. brain heart infusion, and the bacterium to be cultured is Neisseria, it can be desirable to add inducer to the medium after addition of bacteria and the iron and further chelator, preferably about 5 to 20 hours later, e.g. about 7 to about 10 hours later. When the growth medium is less nutrient rich, e.g. a DMEM or a defined growth medium, inducer can be added to the growth medium from about 7 to about 30 hours after addition of bacteria and iron and further chelator, e.g. about 10 to about 20 hours after.

Where the medium is to contain inducer bound to iron and be substantially free of unbound iron, a complex of iron and inducer can be added to the culture medium at the start of bacterial growth, e.g. at about the same time as the bacterial inoculum.

A complex of iron and inducer can be produced by mixing the inducer with an excess amount of free iron, e.g. in a molar ratio of about 1 to 2 of inducer to iron. Unbound iron can be removed from the complex by per-se conventional methods, for example by differential solubilisation.

Where the medium is to contain a low level of iron, e.g. less than about 1 microM, e.g. about 3-6 nanograms per ml, it is sometimes preferred to add inducer to the medium at the start of bacterial growth, e.g. at about the same time as the bacterial inoculum.

Where the medium initially contains sufficient iron for iron-unlimited growth, e.g. more than about 1 microM iron, inducer is preferably added to the medium at a later stage of bacterial growth, e.g. when the bacteria have reached late-log phase.

The range of bacteria to which the present invention can be applied is wide: e.g. to bacteria whether gram-positive or gram-negative which make siderophores and/or have catechol-uptake pathways. Examples include Neisseria, E. coli, and other bacteria such as for example those mentioned in WO 98/56901 (cited above).

The bacteria used herein can for example be vaccine strains derived from Neisseria meningitidis, e.g. of Group B or C, e.g. the Group B strain B16B6 strain of Neisseria meningitidis, or from E. coli, for example E. coli of type 0157 or of a type antigenically related to and cross-reactive with the characteristic antigens of E. coli 0157. Bacteria used in the present invention, such as for example Neisseria or E. coli, can be of attenuated strains, for example Neisseria meningitidis or E. coli with at least one per-se known ‘aro’ mutation, e.g. an “aro B” mutation, or other mutation rendering them auxotrophic. When the bacteria are “aro B” mutants or other auxotrophs it is necessary to add to the growth medium the corresponding required accessory substance e.g. L-phenylalanine, L-tyrosine and L-tryptophan in an amount sufficient to provide for the growth needs of the auxotrophic mutant. The bacteria can also usefully comprise at least one further per-se known mutation, for example, e.g. in Neisseria, a mutation in a gene encoding the bacterial capsule, recombinase, lipid A and/or GalE.

The methods described herein can be applied to a bacterial strain in which expression of the functional fur (ferric uptake regulation) gene product is downregulated, e.g. a regulatory fur mutant strain, as described in WO 98/56901 (cited above), e.g. a regulatory fur mutant with a very low basal level of expression of the fur gene product, as in WO 98/56901. In other useful embodiments, the bacterial strain can be other than a regulatory fur mutant in which the expression of the fur gene product (or its homologue) is regulated independently of the iron concentration in the environment of the bacterium.

Antigen preparations made for vaccine use as described herein can be for example live attenuated bacterial vaccine preparations; or preparations of bacterial vesicles isolated from bacterial cultures, e.g. free of viable live bacteria; or preparations of bacterial outer membrane or cell wall or other antigenic compositions obtained from the cultured bacteria, e.g. free of viable live bacteria; and otherwise sterile and made under pharmaceutically acceptable conditions.

Also provided by the invention is a culture medium for culturing bacteria to make an immunogenic preparation e.g. a live attenuated or a vesicle based vaccine, the medium being characterised by containing an amount of at least one catechol-group-containing inducer agent able to interact with the bacterial catechol-uptake pathway to promote expression of at least one inducible antigen as mentioned herein, e.g. containing norepinephrine at a concentration of for example about 10⁻⁵ to 10⁻³ M. The medium can be in a sterile liquid form, or as a dried or condensed composition of components for constituting such a medium. The medium can also have any of the further features disclosed elsewhere herein.

Such a culture medium can be based on a known medium, for example in the case of culture of Neisseria, the Modified Catlin medium described in J Fu et al, Bio/Technology vol 13 (Feb 1995) pp 170-174, and references cited therein, which can be adapted for present purposes by adding an inducer agent as discussed herein. Culture media for other bacteria can in similar manner be readily adapted from per-se known culture media for the respective bacterial type.

In certain useful embodiments, the bacterial culture can be commenced at low bacterial inoculum level, e.g. in the range of 50-1000 bacterial cells/ml, e.g. 50-250 cells, or fewer. This can be especially desirable when the bacterium to be cultured is E. coli. However, when the bacterium to be cultured is Neisseria, it is often preferable to start the bacterial culture from about 1×10³ to about 1×10⁵ cfu per ml, e.g. about 1×10⁴ cfu/ml: but higher inoculum levels can be used if desired.

The present inventors find that conventional non-catechol iron-chelators, such as EDDA, do not reliably allow good growth of Neisseria under low-iron conditions, nor to a lesser extent do they allow good growth of E. coli under low iron conditions and low inoculum levels such as those just described above. Particularly in embodiments which involve E. coli it is sometimes considered less desirable to use high inoculum levels, which in the case of E. coli can encourage growth in culture with EDDA. The use of norepinephrine or another of the inducers mentioned herein can contribute to solve the problem of producing good growth at low initial inoculum levels.

In the presence of such inducer agents, induction or promotion of growth can occur, and the Neisseria or other bacteria so grown can contain a useful pattern of antigen expression which is qualitatively or quantitatively different from the pattern of antigen expression of corresponding bacteria grown in conventional culture media. For example, with Neisseria or E. coli, the presence of proteins that can be upregulated as described herein can be detected by the use of Coomassie staining of SDS-PAGE electrophoretograms from the bacterial protein mixtures obtained from the cultured bacteria. This comparison can be made for example between a culture grown as described herein with norepinephrine, and a culture made with a conventional iron-chelator such as EDDA but without inducer of the kind discussed herein, and with a culture raised in iron-replete conditions. The upregulation of proteins can be shown for example by extra, or intensified, protein bands in the culture according to the invention. The additional or intensified bands can correspond to proteins with molecular weights for example in the range of about 60-200 kDa, e.g in the range 60-150 kDa.

Similar results in the induction of growth and promotion of a characteristic pattern of antigen expression can be achieved with a wide variety of non-toxic catechol-group-containing iron-chelators compatible with the culture of Neisseria or other bacteria such as E. coli.

It is believed that norepinephrine and others of the inducing agents have effects on the bacterial cells by activating genes for proteins that are not simply responding to the iron status of the medium: culture as described herein is believed to lead inter alia to the expression of immunogenically useful outer membrane proteins—some of which appear not to be iron-regulated proteins.

It is believed that a number of useful antigens can be expressed in response to culture in media containing the inducers described herein, and accordingly the useful properties of the preparations described herein are not limited to those arising directly from presence of the inducible iron-uptake proteins.

Also provided by an aspect of the invention is a preparation of bacterial immunogens, e.g. from Neisseria or from or E. coli, obtainable by processes as described herein, and containing an immunogenic amount of at least one antigen inducible by norepinephrine. The preparation can in certain embodiments be a preparation derived from a regulatory fur mutant in which the expression of the fur gene product (or its homologue) is regulated independently of the iron concentration in the environment of the bacterium, or it can be derived from a bacterial mutant strain other than a regulatory fur mutant, e.g. an iron biosynthetic, metabolic or uptake mutant.

One example of a method for culturing Neisseria for vaccine purposes according to the present invention comprises culturing an attenuated mutant of Neisseria meningitidis, e.g. an auxotrophic mutant such as an aro mutant, under culture conditions as indicated in U.S. Pat. No. 5,597,572 (Centro Nacional de Biopreparados: C C Huergo et al) with the modification that an inducer such as norepinephrine is present in the culture medium, e.g. in an amount as indicated herein, along with (in the case of an auxotrophic mutant) an amount of a nutrient such as tyrosine, tryptophan or phenylalanine corresponding to what is made deficient by the auxotrophic mutation and sufficient to provide for the culture needs of the auxotrophic mutant.

Among the preparations provided by the invention are live attenuated vaccines containing Neisseria cultured in the manner described herein, and preparations of membrane vesicles or blebs or outer membrane proteins derived from Neisseria cultured in the manner described herein.

A preparation of Neisseria antigens obtainable from a culture raised under conditions sufficient to derepress inducible genes and its corresponding protein product, can be identified for example as follows:

Antiserum can be raised in per-se known manner against Neisseria antigens, e.g. a bacterial culture of Neisseria.

Antibody that can distinguish antigens of the inducible Neisseria iron-uptake system can be made by the following steps:

(a) Raise antibody in per-se known manner against a preparation of Neisseria antigens from a Neisseria culture which has been grown with norepinephrine, according to the present invention.

(b) Absorb the antibody in per-se known manner against a preparation of Neisseria antigens from a Neisseria culture grown under conditions of limiting iron supply without inducer.

For the purposes of an example of a suitable test material, a culture produced in low-iron culture conditions can be produced by growing Neisseria in a standard medium containing sufficient iron for growth, and then, after culture, when the biomass of bacteria has increased for example to more than about half (e.g. about 90%) of the total biomass that would be available from that culture if continued to maximum growth without interruption, adding to the culture a sufficient amount of iron-chelator (such as EDDA) (but not any of the inducers described herein) to produce low-iron conditions, including an amount of inducer according to the invention, and then further incubating the bacterial culture, e.g. for a few hours or overnight, to allow the bacteria of the culture to express proteins characteristic of iron-limiting growth conditions.

Sufficient iron for growth of Neisseria etc can be for example of the order of 50 ng/ml Fe (ferrous or ferric iron), of the order of 1 micromolar, and up to about 200 ng/ml or more.

A suitable iron-chelator to produce low-iron conditions for the purposes of this specification can be added at, for example, about 4 times the molar amount of the initial iron content of the culture medium. For the purposes of producing comparative test material such a chelator can be for example EDDA (ethylene diamine di-(ortho-hydroxy-phenylacetic acid)) or under certain conditions desferral.

A further method for distinguishing the presence of proteins that can be upregulated by culture according to methods of the invention as decribed herein comprises the use of comparative SDS-PAGE analysis (with Coomassie staining) of relevant strain of bacteria (or of corresponding bacterial outer-membrane preparations).

SDS-PAGE analysis of bacteria cultured (a) under normal (iron-rich conditions) without the agents described herein, and (b) under low-iron conditions with EDDA, and (c) according to an example of the invention, under low-iron conditions in the presence of epinephrin or another of the agents described herein, shows protein bands, e.g. in the 60-150 kDa size range, that are associated with culture according to the present invention but not with the other culture conditions.

The invention, and materials and methods applicable to carrying out embodiments thereof, is further illustrated, but without intent to limit its scope, by the following description and accompanying drawings, which are described in further detail below:

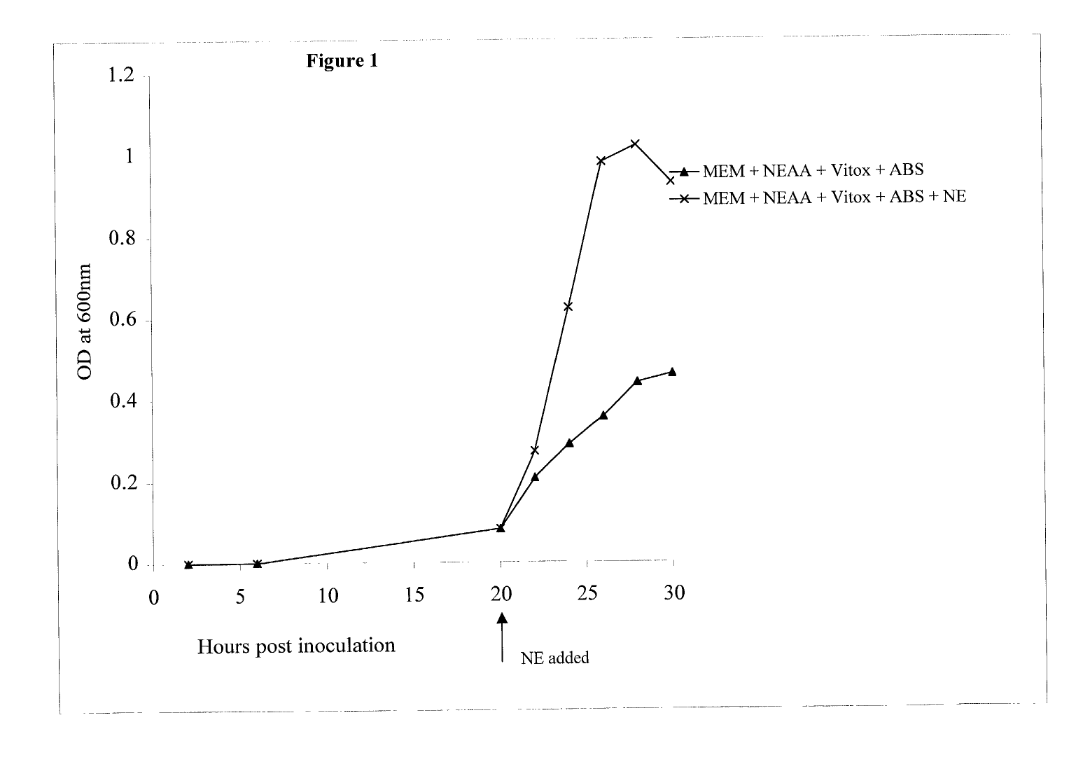

FIG. 1 is a graph showing growth of Neisseria meningitidis in the presence (and for comparison the absence) of norepinephrine bitartrate.

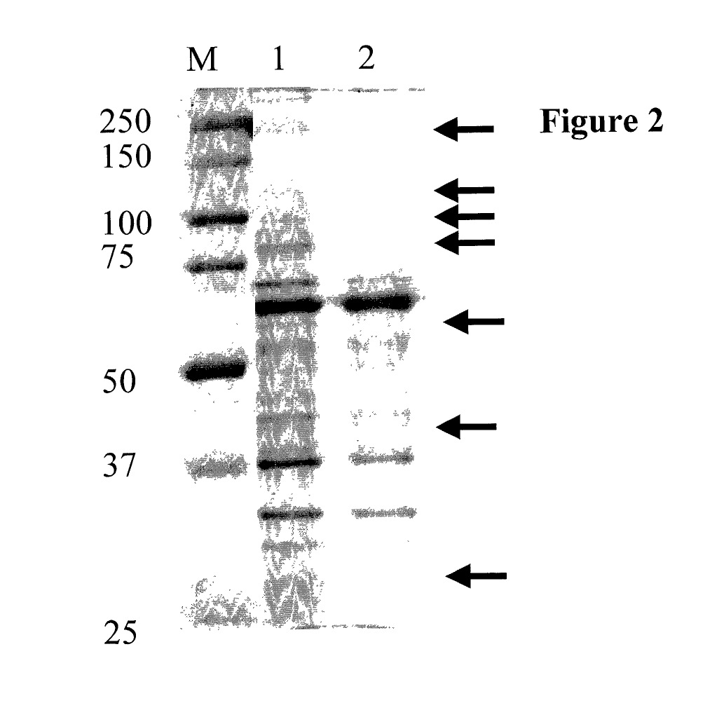

FIG. 2 shows SDS-PAGE gel separation of proteins from Neisseria meningitidis cultured in the presence (and for comparison the absence) of norepinephrine bitartrate.

An experiment carried out as follows illustrates accelerated growth of a culture of Neisseria meningitidis due to addition of norepinephrine bitartrate.

Inoculum preparation:

A culture of Neisseria meningitidis strain B16B6 (obtainable from the National Collection of Type Cultures, PHLS, Colindale, London, UK) was grown overnight at 37 deg C. on a GC agar plate (Oxoid) containing 2% Vitox (Oxoid). Vitox is a defined growth supplement suitable for enrichment of culture media and contains, amongst other ingredients, a source of iron.

Vitox contains (per vial after reconstitution): vitamin B12 (0.1 mg), L-glutamine (100 mg), adenine SO4 (10 mg), guanine HCl (0.3 mg), p-aminobenzoic acid (0.13 mg), L-cystine (11 mg), NAD (Coenzyme I) (2.5 mg), cocarboxylase (1 mg), ferric nitrate (0.2 mg), thiamine HCl (0.03 mg), cysteine HCl (259 mg), and dextrose (1 mg).

At the end of incubation, about 20 bacterial colonies were harvested from the GC agar plate and suspended in 100 microliters of sterile phosphate-buffered saline (PBS; Gibco). The resulting bacterial suspension was then grown at 37 deg C for 4 hours on a GC agar plate containing 2% Vitox. At the end of incubation, the agar plate was flooded with 2 ml of sterile PBS and the bacterial suspension transferred into a sterile container. Optical density of the suspension was then determined at 600 nm. An optical density at 600 nm of 0.1 is equivalent to about 2×10⁸ cfu/ml of Neisseria meningitidis.

Culture in the presence of norepinephrine barbiturate:

The bacterial suspension obtained above was diluted in minimal essential growth medium without any added phenol red indicator (MEM; Gibco) and containing 1% v/v of non-essential amino acid solution (NEAA; Gibco) and 20 mg of magnesium chloride, to yield a suspension containing 1×10⁶ cfu per ml of Neisseria.

Two hundred microliters of the above bacterial suspension was added to 20 ml of the following test medium:

Test medium: MEM (minus phenol red), and containing 1% NEAA, 200 mg magnesium chloride, 0.1% Vitox and 5% adult bovine serum (ABS; Autogen Bioclear, Wiltshire, UK).

The test medium was incubated at 37 deg C. with shaking at 180 rpm. The optical density of the culture was measured at 600 nm at 2, 4 and 20 hours post-inoculation of the media.

Addition of norepinephrine bitartrate:-

At 20 hours post-inoculation, the test sample was divided into halves. 100 micromolar (final concentration) of norepinephrine bitartrate (Sigma) was added to one half of the sample. Both half-samples were then further incubated at 37 deg C., and optical density at 600 nm measured every 2 hours until 30 hours post-inoculation.

Protein analysis of bacteria grown with and without norepinephrine bitartrate:

Samples of “test” bacteria grown as described above in MEM containing NEAA, iron chelator (ABS) and iron (Vitox) both in the presence and absence of norepinephrine bitartrate were taken. The samples were centrifuged to pellet bacteria. The bacterial pellets obtained were resuspended in 50 microliters of TE buffer (Sambrook et al: 1989, Molecular cloning: A Laboratory Manual, Cold Spring Harbour Laboratory Press, N.Y.) and 200 microliters of SDS-PAGE solubilisation buffer added (Sambrook et al: as above). The mixture was then heated at 70 deg C to solubilize proteins. Samples and appropriate molecular weight markers were then run on 10% and 12% SDS-polyacrylamide gels. The gels were then stained with Coomassie blue stain, according to a standard method, and protein bands observed.

In FIG. 1 (a graph showing growth of Neisseria meningitidis in the presence and absence of norepinephrine bitartrate as described above), data points indicated by crosses show bacterial growth in MEM (minimal essential medium) containing NEAA (non-essential amino acid solution), Vitox (a defined growth supplement suitable for enrichment of culture media (Oxoid, components are described below)), ABS (adult bovine serum: an iron chelator), and NE (norepinephrine bitartrate: Sigma). Data points indicated by triangles show bacterial growth in MEM containing NEAA, Vitox and ABS, but without any NE.

FIG. 2 shows SDS-PAGE gel separation of proteins from Neisseria meningitidis cultured with and without norepinephrine. Culture was carried out in MEM medium containing NEAA, iron (Vitox) and an iron chelator (ABS), either with norepinephrine bitartrate added to the culture (lane 1), or without norepinephrine bitartrate (lane 2). Molecular weight markers (kD) are also shown (lane M). In FIG. 2, comparison of protein bands respectively present in lanes 1 and 2 reveals the presence of upregulated or new proteins in Neisseria cultures grown in the presence of norepinephrine (lane 1) which are present in lesser amounts or undetectable after growth in the absence of norepinephrine (lane 2).

In particular it is seen that Neisseria grown in the presence of norepinephrine bitartrate contained expressed proteins, including proteins with molecular weights of about 100 KDa and about 55 KDa, which were not expressed when bacteria were grown in the absence of norepinephrine bitartrate. It has also been seen that when bacteria were grown in the presence of norepinephrine bitartrate, there was over-expression of an approximately 20 KDa protein in comparison to the amount of this protein in bacteria grown without norepinephrine bitartrate. In addition it has been seen that the bacteria cultured with norepinephrine contained either newly expressed or overexpressed further proteins with molecular weights of about 250 kDa, about 80 kDa, and about 28 kDa, by comparison with the proteins of the bacteria cultured without norepinephrine.

Bacterial cultures grown as described above in the presence of an iron source (Vitox) and an iron chelator (ABS), and with addition of norepinephrine bitartrate, can usefully be used in vaccine formulations. In other embodiments the bacteria cultured as described above can be of other strains, e.g. attenuated mutant strains such as auxotrophic mutants, e.g. aro mutants, and/or fur mutants.

Bacteria grown as described can be harvested from the culture medium e.g. by centrifugation and then washed and formulated in a suitable buffer such as a buffer for intranasal administration, e.g. containing phosphate and sucrose and/or trehalose and/or glycerol and/or soya peptone. The bacteria so formulated can be stored frozen e.g. at 80 deg C. or −20 deg C., or they can be lyophilized before storage. Aseptic conditions are used as appropriate.

Live attenuated bacteria grown as described herein can be administered to a human or non-human animal subject to elicit an immune response, e.g. by intranasal, subcutaneous or intramuscular administration. Dosage can be in the range about 10⁶ to about 10¹⁰ cfu of bacteria per dose, e.g. from about 10⁷ to about 10⁹ cfu, e.g. about 10⁸ cfu per dose.

Immunization can be carried out either with single doses or with multiple doses, e.g. up to about 4 doses up to about 4 weeks apart, and also optionally a booster dose after about 6 months.

The invention is susceptible of modifications and variations as will be apparent to the reader skilled in the art. Techniques for mutation, culture and antigen preparation as described in the prior art can be combined and adapted with the use of the techniques described herein for making antigen preparations for use as vaccines. The present disclosure extends to all combinations and subcombinations of the features mentioned or described herein including those in the appended claims. Documents cited herein are incorporated by reference in their entirety for all purposes.

We claim:

1. A method of producing a bacterial antigen preparation which comprises the steps of (a) culturing bacteria that possess a catechol-uptake pathway, in a medium which comprises a catechol-group-containing inducer of bacterial growth, whereby said inducer stimulates growth of said bacteria, (b) harvesting from said culture a bacterial antigen, and (c) formulating said antigen with a pharmaceutically acceptable carrier for use as an immunogen.

2. A method according to claim 1, wherein said bacteria are of a strain of Neisseria.

3. A method according to claim 2, wherein said bacteria are of an attenuated mutant strain of Neisseria meningitidis.

4. A method according to claim 3 wherein said bacteria are of an auxotrophic aro mutant strain of Neisseria, wherein said Neisseria has at least one further attenuating mutation.

5. A method according to claim 1, wherein said catechol-group-containing inducer comprises norepinephrine.

6. A method according to claim 1, wherein said medium further comprises an iron chelator which does not contain a catechol group.

7. An composition comprising a bacterial protein from Neisseria or E. coli, wherein said bacterial protein, obtained by culturing Neisseria or E. coli by the method according to claim 1, said preparation comprising at least one bacterial protein which is induced by norepinephrine.

8. The composition according to claim 7, wherein said bacteria are Neisseria and wherein said preparation comprises at least one bacterial protein induced by norepinephrine and wherein said bacterial protein has a molecular weight of about 50 kDa, or about 55 kDa, or about 20 kDa.

9. The composition according to claim 8, wherein said bacterial protein is in a live attenuated bacteria, in an isolated bacterial vesicle, or is an isolated bacterial outer membrane protein.

10. A method of evoking an immune response against a bacterium in a subject, comprising administering to said subject the composition according to claim 7.

11. The method of claim 1, wherein said bacteria is selected from the group consisting of Neisseria and E. coli.
